(19) United States
(12) Patent Application Publication
(10) Pub. No.: US 2016/0100775 A1
(43) Pub. Date: Apr. 14, 2016

(54) Integrated Movement Assessment System

(71) Applicants: Mary Reaston, Carlsbad, CA (US); Phil Reaston, Carlsbad, CA (US)

(72) Inventors: Mary Reaston, Carlsbad, CA (US); Phil Reaston, Carlsbad, CA (US)

(21) Appl. No.: 14/512,438

(22) Filed: Oct. 12, 2014

(51) Int. Cl. A61B 5/11; A61B 5/0488; A61N 7/00; A61F 7/00; A61N 1/36; G06F 19/00; A61B 5/00

(52) U.S. Cl. CPC A61B 5/1114; G06F 19/3418; A61B 5/0488; A61B 5/0015; A61B 5/1126; A61F 7/00; A61N 1/36139; A61N 7/00; A61F 2007/0094

(57) ABSTRACT

An integrated movement assessment system (IMAS) that monitors a subject's movement by utilizing electromyography (EMG), range-of-motion (ROM) and functional capacity assessment (FCA). The EMG, ROM and FCA is used in combination with proprietary software that acquires data from wireless sensors which are attached to the subject and are capable of transmitting data wirelessly. In addition to assessment, the IMA can perform baseline and post-loss comparisons that are specific to a subject's occupation. When necessary or desired, the IMAS can also provide site-specific therapy.

### TECHNICAL FIELD

The invention generally pertains to diagnostic analysis and treatment systems, and more particularly to an integrated movement assessment system that monitors a subject's movement by electromyography (EMG), range-of-motion (ROM) and functional capacity assessment (FCA), and that can also provide therapy based on the EMG, ROM and ECA.

### BACKGROUND ART

Unresolved injuries represent a significant medical problem today, with back pain alone accounting for the largest medical visits. Carpal tunnel syndrome (CTSs) and repetitive stress injuries (RSI) account for the most days lost and are predicted to become the most costly health problem of our time. The U.S. Department of Labor and Occupational Safety and Health Administration (OSHA) define a musculoskeletal disorder (MD) as an injury if the muscles. nerves, tendons, ligaments, joints, cartilage and spinal discs. OSHA identities examples of MSDs to include: Carpal tunnel syndrome, Rotator Cuff syndrome, De Quervain's disease, Trigger finger, Tarsal tunnel syndrome, Sciatica, Epicondylitis, Tendinitis, Raynaud's phenomenon, Carpel layers knee, herniated spinal disc, and Low back pain.

These types of disorders commonly referred to as soft tissue injuries (STI) as well as sprains and strains most often present as injury or pain of the back, neck, shoulder or knee, are a major source of disability. Taken together, they represent the majority of compensable injuries accounting for 29% of total work related injuries. According to OSHA, the average cost per incidence of an MSD is estimated to be $12,000. If surgery is required, the average cost rises to $43,000 per incidence according to the American Society of Orthopedic Surgeons. MSDs cost U.S. industry $15-20 billion in worker's compensation costs with total costs as high as $45-60 billion per year.

While frequently utilized, subsequent diagnostic modalities are, in many cases, not appropriate for assessing tissue injuries.

X-ray investigation can be used to assess the possibility of fracture or dislocation: however, in low back pain (LBP) x-ray is rarely indicated. Nerve conduction studies may be used to localize nerve dysfunction, and Electrodiagnosis may help differentiate between myopathy and neuropathy. Magnetic resonance imaging (MRI) and CT scans, while excellent tests to evaluate structure are static and not designed to assess muscle function dynamically (while patient is in motion). In addition, these standard tests all carry a high false positive rate. The results provided by these modalities are subject to different interpretations and may be inaccurate and inconclusive. Despite these shortcomings, 1 to 3 Medicare beneficiaries receive an MRI of their lower back when they complain of pain, rather than trying more recommended—and potentially safer—treatment first, such as physical therapy. Not surprisingly, soft tissue injuries are difficult to diagnose because the above diagnostics are frequently unable to document the presence of pain and loss of function. In many instances, this leads to prolonged duration of disability and lost time, and increased medical costs, based on poorly defined diagnosis and no classification of work category. This can leads to costly misdiagnosis, unnecessary surgery, prolonged treatment periods, and fraudulent claims. In the absence of objective medical evidence, “proof” of a soft tissue injury is typically established through medical records documenting results of medical examinations and the insured's complaints of pain and in cases of litigation, testimony from dueling experts whereby each party presents a medical opinion. The need for accurate, timely and evidence-based diagnosis and treatment for soft tissue injuries is needed to curtail these escalating costs and improve clinical outcomes.

A study in the New England Journal of Medicine indicates that over 58% of asymptomatic low back pain patients who underwent an MRI found evidence of disc pathology. How reliable is an MRI—it appears to have no correlation to pain, impairment and may not be clinically significant for soft tissue injuries.

A recent study revealed that over 45 percent of individuals who have undergone CTS release surgery had no improvement two years past the surgical intervention because they were misdiagnosed. The individuals probably had cervical pathology that can refer pain and mimic the symptoms of carpal tunnel, ulnar neuopathy, cubital tunnel, tendonititis, DeQuarians syndrome I.E., repetitive stress injuries. The problem is that until the development of the instant invention, there was no way to ascertain if the problem was proximal (cervical or distal CTs).

The inventive integrated movement assessment system (IMAS) is a portable, non-loading electronic instrument that simultaneously monitors muscle activity, With range of motion and FCA and can also provide site specific treatment The IMAS utilize wireless sensors with a computer and proprietary software to measure EMG, Range-of-Motion and FCA (functional capacity assessment), which can all be measured at the same time.

The IMAS is portable and can be battery operated to allow a patient to be monitored anywhere including at work sight, at home and performing any activity even their job, no matter what or where it is. The IMAS also complies with the ADA law and includes a special filter system to pick up ischemic changes. This is important because it can correlate to disc pathology or spinal changes on an MRI or decreased blood flow to a muscle. Since the IMAS monitors active range of motion, it takes the MRI one step further and can help determine if the monitored ailment, can in fact, be treated with conservative methods that do not involve surgery.

Furthermore, 65 percent of individuals who have undergone back surgery resulted in failed back syndrome or have no relief of symptomology. This can also be attributed to an inability to properly diagnose and age a soft tissue injury. In the past, some prior art has not been capable of performing a proper diagnosis.

Until the advent of the IMAS there was no way to incorporate all of functions required to perform a comprehensive diagnostic test in a reliable, cost effective and user-friendly manner. The IMAS utilizes remote and wireless sensor(s) which can provide portability and ease of use. The wireless sensor(s) can have a disposable component, and any proprietary sensor(s) that are not disposable have their own battery-recharging unit. This allows for no down time during monitoring and, since the sensor(s) can be interchangeable, if one sensor(s) is not functioning, a replacement is immediately available. Therefore, an unlimited combination of testing possibilities is available depending on the protocols required. The IMAS is operated remotely, by utilizing GiFi, WiFi, Zipbee, BLUETOOTH®, or any other standard or proprietary wireless system. The unique wireless technology also does not use pairing to connect so it also allows the use of a larger number of sensors, provides improved sensor monitoring, and increases sensor performance.

It should be noted that in view of current technology, especially medical technology, the use of wireless sensors could not be anticipated. It is true that wireless sensors have been in use for some time, but medical technology has not adopted the use of wireless sensors, and instead has relied on conventional wired sensors. Therefore, even though wireless sensors have been available, the medical community has chosen not to use them, and it could not be anticipated that this is going to change in the future.

Additionally, the IMAS's use of the proprietary software in combination with the wireless sensors that utilize a wireless connection protocol which does not require conventional wireless pairing absolutely discloses a unique medical device that is significantly advanced and completely different from any prior art.

A search of the prior art did not disclose any literature or patents that read directly on the claims of the instant invention. However, the following U.S. patents are considered related:

| PAT. NO. | INVENTOR | ISSUED |
|---|---|---|
| 4,586,515 | Berger | May 1986 |
| 4,667,513 | Konno | May 1987 |
| 4,688,581 | Moss | August 1987 |
| 4,800,897 | Nilsson | January 1989 |
| 4,928,709 | Allison et al | May 1990 |
| 4,938,476 | Brunelle et al | July 1990 |
| 5,042,505 | Meyer et al | August 1991 |

The U.S. Pat. No. 4,688,581 disclosed an apparatus and a method for non-invasive in vivo determination of muscle fiber composition. The method includes the steps of electrically stimulating a chosen muscle; determining the stimulation current; measuring the electrical potential of the muscle; the contraction time; and the force produced by the contraction; and by intercorrelating the data by multiple regression, determining the type, percentage and size of muscle fibers within the muscle stimulated. Apparatus for determining the muscle composition includes a muscle stimulator of controlled voltage; electromyogram equipment; and a force transducer providing a tension curve as well as force measurements.

The U.S. Pat. No. 4,667,513 discloses an apparatus and a method for estimating the degree of the fatigue and pain of muscles. The apparatus composes subjects of different weights on the same basis by deriving the variation in the muscular strength such as the dorsal muscular strength, should muscular strength, the grasping power, and the like. An analogous electric signal integrated the muscular output on one hand, and provides an integrated value of the electromyogrammatic amplitude by processing the voltage induced from the muscle to be tested through an electromyogram amplitude and a waveform processor. The ratio between these integrated values, after correcting the ratio with a weight/muscular strength coefficient is digitally displayed.

The U.S. Pat. No. 5,042,505 discloses an electronic device for measuring relative angular positional displacement and angular range-of-motion for body segments and articulating joints of the human skeleton. The device has a hand-held interface unit which is placed against the body segment or joint to be tested. Mounted within the housing of the interface unit is a shaft with a pendulum at one end and an optical encoder at the other. As the body segment rotates or the joint articulates, the pendulum swings in the direction of gravity, causing the shaft to rotate. The optical encoder generates an electrical signal representative of the amount of rotation of the shaft. The generated signal is fed to a microprocessor which processes the information and can produce on a display the change in angular position relative to initial angular position or the angular range-of-motion of the body segment or articulating joint.

For background purposes and indicative of the art to which the invention relates, reference may be made to the following remaining patents found in the patent search.

| PAT. NO. | INVENTOR | ISSUED |
|---|---|---|
| 8,680,991 | Tran | 25 Mar. 2014 |
| 8,568,312 | Cusimano | 12 Mar. 2010 |
| 8,535,224 | Cusimano | 18 Feb. 2010 |
| 8,449,471 | Tran | 28 May 2013 |
| 8,306,635 | Pryor | 6 Nov. 2012 |
| 7,840,031 | Albertson et al | 23 Nov. 2010 |
| 6,678,549 | Cusimano | 15 Nov. 2001 |
| 5,513.651 | Cusimano | 4 Nov. 1994 |
| 5,056,530 | Butler et al | 15 Oct. 1991 |
| 5,050,618 | Larsen | 24 Sep. 1991 |
| 5,038,795 | Roush et al | 13 Aug. 1991 |
| 5,012,820 | Meyer | 7 May 1991 |
| 4,886,073 | Dillon et al | 12 Dec. 1989 |
| 4,845,987 | Kenneth | 11 Jul. 1989 |
| 4,834,057 | McLeod, Jr. | 30 May 1989 |
| 4,805,636 | Barry et al | 21 Feb. 1989 |
| 4,742,832 | Kauffman et al | 10 May 1988 |

### DISCLOSURE OF THE INVENTION

The integrated movement assessment system (IMAS) combines electromyography with range of motion and functional capacity assessment measurements to provide doctors and other clinical practitioners with a method for accurately analyzing myofascial and other injuries. The system in its basic form is comprised of an computer, at least one wireless range-of-motion (ROM) sensor, and at least one wireless functional capacity assessment (FCA) sensor. The IMAS utilizes proprietary software for protocols and the wireless connection(s) can be facilitated by various wireless connection methods.

The device is a portable, non-loading electronic system that incorporates electromyography (EMG) that utilizes at least one EMG sensor, at least one range of motion sensors, and at least one functional capacity assessment sensor

The simultaneous monitoring of the muscle groups allowed by the IMAS measures muscle tone, muscle spasms, muscle activity and response, as well as muscle recovery ischemic activity and fatigue. This is accomplished for each muscle group monitored while several muscles are being monitored at the same time above and below the area of complaint. This allows an analyst with the system, to outline a specific therapy program for the problem and traces the referred pain problem. With a site specific therapy protocol, physical therapy is reduced to 50-60 percent less sessions, decreases costs, treatment time and directs the specific type of treatment like electrical stimulation, ultra sound, massage, heating or cooling to a specific location. Thus, medical costs related to treatment and use of medication are greatly reduced.

In view of the above disclosure, the primary object of the invention is to provide doctors and other diagnostic personnel with a portable, wireless system that simultaneously utilizes electromyography in combination with range of motion and functional capacity assessment to monitor any muscle group(s) in the human body and can be used to provide site specific therapy.

In addition to the primary object, it is also an object of the invention to provide a system that:
measures compliance without a subject's cooperation. Because the ROM and FCA can be combined with specific EMO readings, the system can tell if a subject cannot complete the range of motion or a lifting task. This is very important to the insurance industry to reduce and defer fraudulent worker's compensation and personal injury claims,
includes a specific protocols for example for carpal tunnel syndrome (CTS) that monitors the testing and range of motion readings for all cervical and upper extremity muscle groups. This interactive protocol with the system allows doctors to look at the relationship between muscle groups and to diagnose if the problem is cervical CTS or cubital tunnel. The system also allows doctors to determine if it is a repetitive stress injury. Also includes baseline and post loss protocols that comply with ADAAA, EEOC and job functions,
is beneficial to sports in that it can tell an athlete what muscle groups to work out with what procedure and for how long before the muscle fatigues; thus, it maximizes the work-out period without causing injury,
is beneficial for post offer pre-employment screening or a compliance of the existing workforce to have a “finger print” of muscle activity if there is a subsequent injury,
can diagnose soft tissue injury,
can determine the age a soft tissue injury,
can objectively determine compliance, malingering or pain,
can tell if disc pathology is present and if it is clinically significant,
can provide site-specific therapy protocols, and
can provide site specific treatment.

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is a block diagram showing an embodiment of an integrated movement assessment system.

FIG. 2 is a block diagram showing the steps performed during an integrated movement assessment.

FIG. 3 is a block diagram showing an embodiment of an integrated movement assessment system.

FIG. 4 is a block diagram showing an embodiment of an integrated movement. assessment system.

### BEST MODE FOR CARRYING OUT THE INVENTION

The best mode for carrying out the invention is presented in terms that disclose a preferred embodiment of an integrated movement assessment (IMAS). The IMAS utilizes electromyography in combination with range of motion and functional capacity assessment, as well as proprietary software to monitor any muscle group in the human body and to provide site-specific therapy.

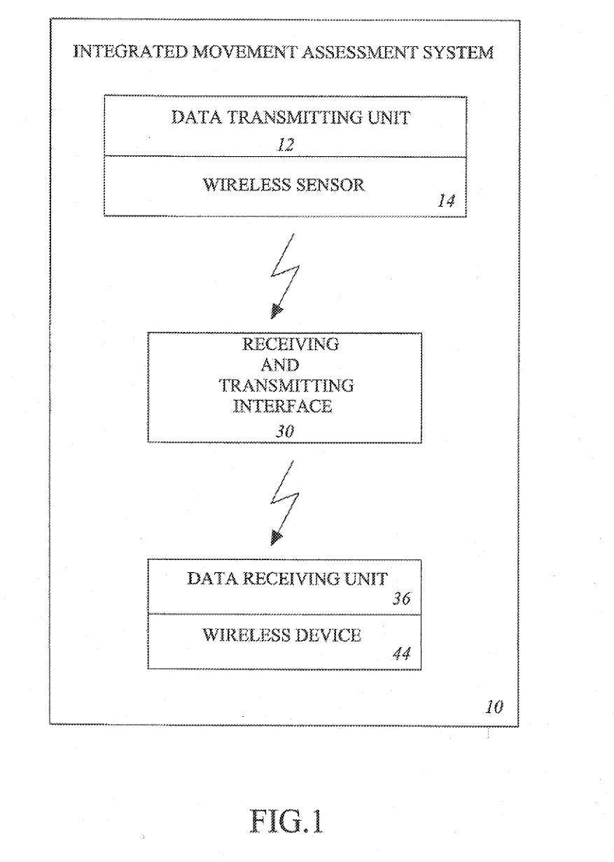

The basic embodiment of the IMAS 10, as shown in FIGS. 1-4, is comprised of the following major elements: a data transmitting unit 12 consisting of at least one wireless sensor 14, a receiving and transmitting interface 30 and a data receiving unit 36.

As shown in FIG. 1, the data receiving unit 36 is comprised of a computer that functions with a proprietary software program 46. The computer is comprised of a wireless device 44 that is selected from the group consisting of a laptop computer, a desktop computer, a tablet computer, or a smart phone. These wireless devices each utilize a wireless protocol that is selected from the group consisting of BLUETOOTH®, GiFi, WiFi, ZipBee, WiMAX, WLAN, WPAN, WBAN, on a proprietary wireless connection means that has been invented by the applicants (U.S. patent application Ser. No. 14/171,373). The software program 46 can also be accessed and utilized by the wireless device in the form of a computer application or APP.

The data receiving unit 36 comprising the computer with proprietary software, as shown in FIG. 1, receives data from the data transmitting unit 12 comprising at least one wireless EMG sensor 18, at least one ROM sensor 20 and at least one FCA sensor 22, all or each of which are connected to a subject. The data from the sensor(s) 14 is wirelessly transmitted to the data receiving unit 36, 44 via a receiving and transmitting interface 30, as shown in FIG. 1. It is important to disclose that the wireless sensor(s) which are utilized in the instant application do not have to be attached to a subject, as most conventional prior art sensors must be. The applicant's wireless sensor(s) merely have to interface with a subject. As long as the wireless sensor(s) are in contact with the subject, the sensor(s) will provide the necessary functionality. For example, the wireless sensor(s) can simply be placed on a subject, with no attachment or adhesion required. This is a significant improvement over conventional prior art sensors.

Each of the least one EMG sensors 18 produces at least one differential signal representative of the resistance between two skin contact points of a subject. Each of the at least one ROM sensors 20 produces at least one range of motion signal representative of the angular distance produced from selected areas of the subject. Each of the least one FCA sensors 22 produces at least one differential signal representative of a pulling force exerted by a subject.

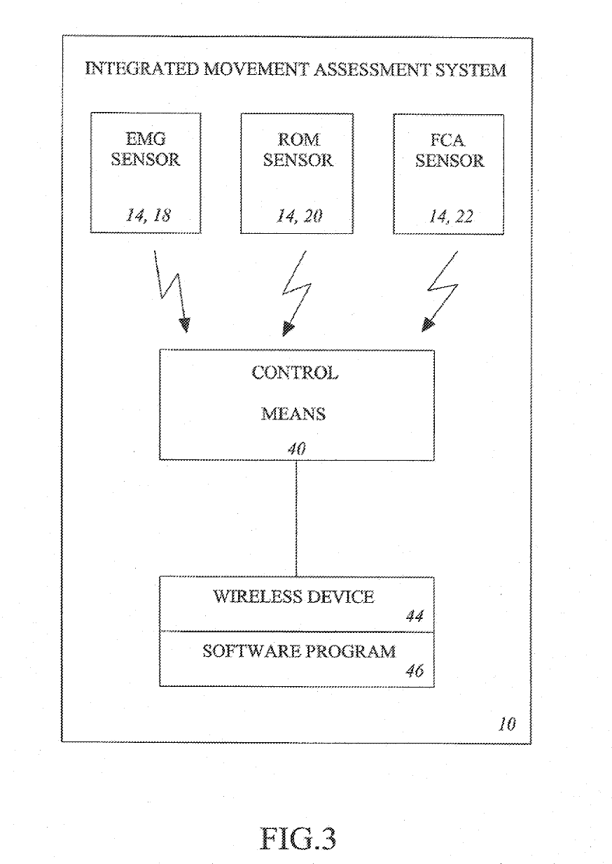

From the respective EMG, ROM and FCA sensors, as shown in FIG. 3, data is wirelessly transmitted to the control means 40 and onto the computer 44. The computer operates with the proprietary software program 46 to produce the comparative analytical data representative of the subject's problem being analyzed. The software program:

a) records patient demographics

b) selects the appropriate testing protocol,

c) samples each cable led during the test to detect if a sensor failure has occurred, or when appropriate information is transmitted

d) prompts the system user as to the location of a sensor failure,

e) starts the computer when the testing should begin,

f) prompts the technician as to the muscle groups that the individual sensors should be connected to for a given protocol,

g) prompts the technician as to the activities that the subject should be performing during the test cycle,

h) sets the testing time for each test being performed,

i) saves the data at the completion of a test,

j) transmits the data, and

k) connects protocols

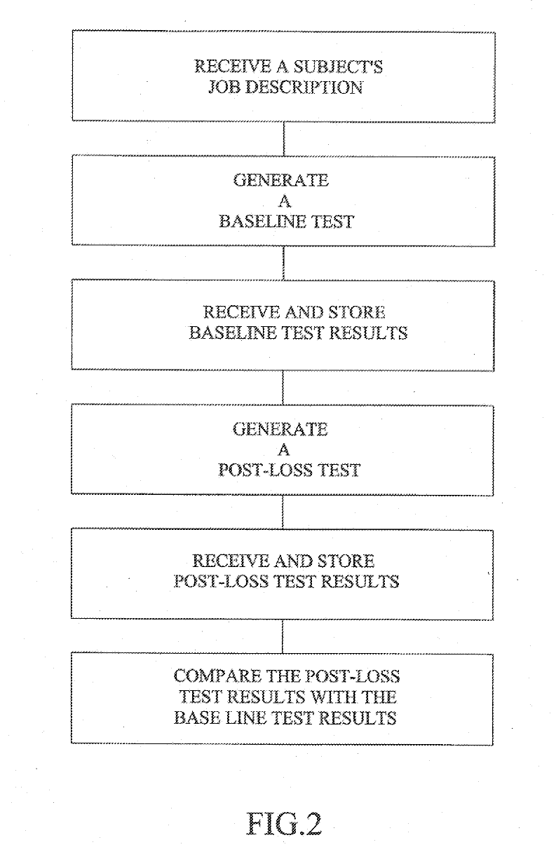

The integrated movement assessment is performed by the following steps, as shown in FIG. 2:
a) receive at a physical or a hosted server a subject's specific job description from an employer for a specific job position,
b) generate a non-loading, non-invasive baseline test that comprises a testing protocol based on the subject's specific job description and specific body parts,
c) receive and store in a memory medium, without interpretation, baseline test results for a subject in the specific job position,
d) generate a post-loss test comprising a testing protocol that matches at least a portion of the testing protocol of the baseline test and is segregated for the subject's specific body parts,
e) receive and store in the memory medium post-loss test results for the employee, and
f) compare the post-loss test results with the baseline test results to determine a difference between the post-loss test results and the baseline test results and to record a comparison results.

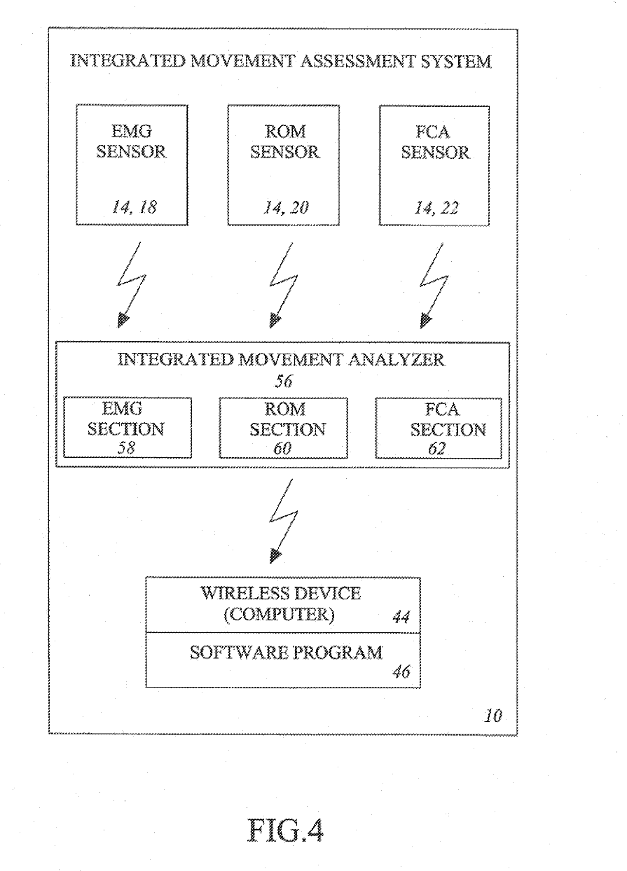

In another embodiment of the IMAS 10, as shown in FIG. 4, the EMG sensor(s) 14, 18; ROM sensor(s) 14, 22 and FCA sensor(s) 14, 22 wirelessly transmit data to an integrated movement analyzer 56 having an EMG section 58, a ROM section 60 and an FCA section 62. Each of the representative EMG data, ROM data and FCA data is applied to the representative EMG section 58, ROM section 60 and FCA section 62. From the integrated movement analyzer 56 the data is wirelessly transmitted to a wireless device 44 which is also known as a computer that is utilizing a software program 46, as shown in FIG. 4.

It should be noted that the integrated movement assessment can be further performed by wirelessly attaching at least one sensor to any selected muscle group(s) and measuring the activity. By use of the wireless sensors, the number of testing protocols are unlimited, and the muscle/muscle groups that can be tested are united only by the number of muscles/muscle groups in the person's body.

Although the IMAS has been disclosed for use on a human subject, the IMAS can also be effectively utilized to perform an integrated movement assessment on an animal subject.

While the invention has been described in detail and pictorially shown in the accompanying drawings it is not to be limited to such details, since many changes and modification may be made to the invention without departing from the spirit and the scope thereof. Hence, it is described to cover any and all modifications and forms which may conic within the language and scope of the claims.

1. An integrated movement assessment system (IMAS) comprising:
a) a data transmitting unit comprising at least one wireless sensor having an input and an output, wherein the input is attached to a selected area of a subject,
b) a wireless receiving and transmitting interface having means for wirelessly receiving the output from said wireless receiving and processing means and producing an output, and
c) a wireless data receiving unit having means for receiving and processing the output from said receiving and transmitting interface and producing data that is indicative of a subject's test results.

2. The IMAS as specified in claim 1 wherein said at least one wireless sensor is selected from the group consisting of at least one wireless electromyography (EMG) sensor, at least one wireless range-of-motion (ROM) sensor and at least one wireless functional capacity assessment (FCA) sensor, wherein said sensors are utilized one at a time or simultaneously.

3. The IMAS as specified in claim 1 wherein at least one wireless sensor(s) delivers site-specific therapy that is selected from the group consisting of electrical stimulation, massage, ultrasound, heating and cooling.

4. The IMAS as specified in claim 1 wherein said data receiving unit having means for receiving and processing the output from said receiving and transmitting interface is comprised of a computer.

5. The IMAS as specified in claim 4 wherein the computer is comprised of a wireless device that is selected from the group consisting of a laptop computer, a desktop computer, a tablet computer and a smartphone.

6. The IMAS as specified in claim 5 wherein the wireless device utilizes a wireless protocol that is selected from the group consisting of BLUETOOTH®, GiFi, WiFi, ZigBee, WiMAX, WLAN, WPAN, and WBAN.

7. The IMAS as specified in claim 4 wherein the computer functions with proprietary software.

8. The IMAS as specified in claim 1 wherein the subject's test results are reviewed with a previously-acquired baseline test, and are compared to a post-loss test.

9. The IMAS as specified in claim 1 wherein said IMAS further comprises a sensor failure detection software section that alerts a system operator when there is not adequate data.

10. The IMAS as specified in claim 1 wherein said assessment is performed by the following steps:
a) receive a subject's specific job description from an employer for a specific job position,
b) generate a non-loading, non-invasive baseline test that comprises a testing protocol based on the subject's specific job description and specific body parts,
c) receive and store in a memory medium, without interpretation, baseline test results for a subject in the specific job position
d) generate a post-loss test comprising a testing protocol that matches at least a portion of the testing protocol of the baseline test and is segregated for the subject's specific body parts,
e) receive and store in the memory medium post-loss test results for the subject, and
f) compare the post-loss test results with the baseline test results to determine a difference between the post-loss test results and the baseline test results and to record comparison results.

11. The assessment as specified in claim 10 further comprising the following steps:
a) receive the baseline test results and post-loss test results as encrypted, raw data, and
b) decrypt the raw data for storage in the memory medium.

12. The assessment as specified in claim 10 further comprising analyzing the difference between the post-loss test results and the baseline test results for trends using a proprietary database.

13. The assessment as specified in claim 10 further comprising issuing a report of results of comparing the post-loss test results and the baseline test results for automated system administration follow-up and tracking.

14. The assessment as specified in claim 1 further comprising the ability to determine if surgically-implanted cervical, thoracic or lumbar hardware should be removed from an individual, wherein the determination is made by utilizing diagnostic data acquired from a test protocol performed by said IMAS, wherein the diagnostic data is selected from the group consisting of EMG, ROM, FCA, muscle data, myofascial data, structural data, nerve data or ischemic data.

15. An integrated movement assessment system (IMAS) comprising:
a) at least one wirelessly connected electromyography (EMG) sensor which measures a subject's myographic, amplitude, frequency and ischemic changes, wherein said EMG sensor is wirelessly connected to an IMAS control means,
b) at least one wirelessly connected range-of-motion (ROM) sensor which measures a subject's lateral movement, flexion, extension and rotation, wherein said ROM sensor is wirelessly connected to the IMAS control means,
c) at least one wirelessly connected functional capacity assessment (ICA) sensor which measures a subject's functional capacity, wherein said FCA sensor is wirelessly connected to the IMAS control means, and
d) a wireless device utilizing to a software program that provides functional control and data control means for said IMAS, wherein said wireless device interfaces with the IMAS control means.

16. The IMAS as specified in claim 15 wherein said integrated movement assessment is performed by the following steps:
a) receive a subject's specific job description from an employer for a specific job position,
b) generate a non-loading, non-invasive baseline test that comprises a testing protocol based on the subject's specific job description and specific body parts,
c) receive and store in a memory medium, without interpretation, baseline test results for a subject in the specific job position generated by an electro diagnostic functional assessment (EFA) unit,
d) generate a post-loss test comprising a testing protocol that matches at least a portion of the testing protocol of the baseline test and is segregated for the subject's specific body parts,
e) receive and store in the memory medium post-loss test results for the subject, and
f) compare the post-loss test results with the baseline test results to determine a difference between the post-loss test results and the baseline test results and to record comparison results.

17. The IMAS as specified in claim 16 wherein said integrated movement assessment is further performed by wirelessly attaching at least one sensor to a subject's selected muscle groups and measuring the electrical activity, wherein the muscle groups and the measurement process comprises the following steps:
(1) at rest,
(2) performing selected range of motion protocols, and
(3) at rest.

18. An integrated movement assessment system comprising:
a) at least one wireless electromyography (EMG) sensor that is wirelessly connected to a subject, wherein each said EMG sensor produces at least one differential signal representative of the resistance between two skin contact points of the subject,
b) at least one wireless range of motion (ROM) sensor that is wirelessly connected to a subject, wherein each ROM sensor produces at least one range of motion signal representative of the angular distance produced from a selected area of the subject,
c) at least one wireless functional capacity assessment (FCA) sensor that is wirelessly connected to a subject, wherein each FCA sensor having circuit means for produces at least one differential signal representative of a pulling force exerted by the subject,
d) an integrated movement analyzer (IMA) comprising:
(1) an EMG section having sensor means for wirelessly receiving and processing the signals from said at least one EMG sensor,
(2) a ROM section having sensor means for wirelessly receiving and processing the signals from said at least one ROM sensor,
(3) a FCA sensor having circuit means for wirelessly receiving and processing the signals from said at least one FCA sensor, and
e) a computer that utilizes a proprietary software program that receives the signals from said IMA for further processing and for the production of data representative of the subject who is analyzed.

19. The IMAS as specified in claim 18 wherein said at least one EMG sensor, said at least one ROM sensor and said at least one FCA sensor are wirelessly connected.

20. The IMAS as specified in claim 18 wherein the computer is comprised of a wireless device that is selected from the group consisting of a laptop computer, a desktop computer, a tablet computer and a smartphone.

21. The IMAS as specified in claim 18 wherein the wireless device utilizes a wireless protocol that is selected from the group consisting of BLUETOOTH®, GiFi, WiFi, ZigBee, WiMAX, WLAN, WPAN, and WBAN.

22. The IMAS as specified in claim 18 wherein the wireless device utilizes a proprietary wireless connection means that facilitates the wireless connection by means of software and does not necessitate the use of conventional wireless pairing.
